(19) United States
(12) Patent Application Publication
(10) Pub. No.: US 2004/0091539 A1
(43) Pub. Date: May 13, 2004

(54) Dermastick thickened ointment

(76) Inventors: Ake Lindahl, Skurup (SE); Johan Gullberg, Lomma (SE)

Correspondence Address: MANELLI DENISON & SELTER, 2000 M STREET NW SUITE 700, WASHINGTON, DC, 20036-3307, US

(21) Appl. No.: 10/292,756

(22) Filed: Nov. 13, 2002

(51) Int. Cl. A61K 9/00; A61K 9/14; A61K 31/573

(52) U.S. Cl. 424/484; 514/179

(57) ABSTRACT

Provided is a solid stick topical composition containing a corticosteroid. The solid stick also contains petrolatum, wax, propyleneglycol and emulsifier. The solid stick is contained in a protective covering so that during application it does not come in contact with the hand holding the stick.

### FIELD OF THE INVENTION

This invention relates to topical formulations in the form of sticks that contain corticosteroids.

### BACKGROUND OF THE INVENTION

Topical treatment with superpotent corticosteroids has become one of the most attractive treatments in psoriasis and eczema. The topical route of administration is superior to the oral route since systemic administration of these steroids leads to unacceptable side effects. The topical route is however not without problems. The superpotent corticosteroids are affecting not only parts of the skin where symptoms are visible but also healthy skin. It is therefore of great importance to restrict application of superpotent steroids to the affected areas.

Currently, the predominant formulation type is ointments or creams. This type of formulation has to be massaged into the skin and as the formulation is heated by the skin the viscosity decreases with the consequence that the preparation is spread over not only the affected area but also over healthy skin. Another aspect is the forced distribution of cream or ointment over the affected area. Normally this is done by hand with the consequence that the hand used for the application will be treated as well. Long term extra lesional application increases the risk for skin atrophy in these areas.

Another problem associated with local, topical, treatment of psoriasis and eczema is the lag time between application and onset of action. A long lagtime will have impact not only on the effect of the treatment but also on patient compliance.

There have been many attempts at solving the above-described undesirable affects of conventional topical applications of steroids. The following documents attempt to solve these problems, but fall short thereof.

In U.S. Pat. No. 4,299,828 a stick for topical use containing corticosteroids is described. In this patent, the corticosteroid is dissolved in oleaginous solvents defined as one or several of the following solvents: castor oil, mineral oil, isostearyl alcohol, isopropyl palmitate, isopropyl myristate, dibutyl sebacate, diisopropyl sebacate, and mixtures thereof. The patent teaches the importance of saturated solutions for maximisation of penetration rate, although no disclosure is made on penetration or efficacy, and includes detailed information regarding suitability of solvent mixtures for specific purposes. The solvents described in this patent are not required in the present application. Furthermore, this patent does not disclose the unexpected advantages of present invention.

D0320616, now expired, teaches a cosmetic stick mass including an oily solvent for low potency corticosteroids. This publication does not mention any effects of the included steroid(s), nor does it discloses the unexpected advantages of the present invention.

In WO98/18472 a stick formulation containing aciclovir for the treatment of Herpetic infections is presented. The publication does not refer to steroid penetration or conditions for including a steroid, and does not disclose the unexpected advantages of the present invention.

IN WO00/28958 the manufacture of an acetylsalicylic acid stick is presented. The composition is designed to optimise the solubility of acetylsalicylate and does not teach the unexpected advantages of the present invention.

In WO00/44347 a general stick formulation is presented. The formulation must contain at least 85% of solvents and water combined. The formulation is thickened using cellulose based thickeners. This application does not teach the unexpected advantages of the present invention.

In U.S. Pat. No. 4,883,792 a stick formulation of a combination of a corticosteroid and an antifungal agent is disclosed. The formulation consists of a solvent system, water and propyleneglycol, emulsifiers, waxes, buffers etc. The wax content is 2 to 5%. This patent does not teach the unexpected advantages of the present invention.

U.S. Pat. No. 5,110,809 teaches that hydrocortisone, a low potency corticosteroid, penetrates faster through the skin if it is presented in a formulation containing 10% water, 80% alcohols (20% ethanol and 60% propyleneglycol) and a cellulose thickener than if it is formulated in creams or ointments. In the present invention, the amount of propyleneglycol is kept far below 80%. This patent does not teach the unexpected advantages of the present invention.

U.S. Pat. No. 5,543,148 discloses a stick composition based on high amounts of solvent, 70 to 80%, in the form of propyleneglycol and a gelling system based on alkali metal salts of fatty acids. The present invention utilizes far less than 70% of propylene glycol. Furthermore the formulation in this patent contains at least 5% of water. Water is not intentionally included in the present invention. This patent also does not teach the unexpected advantages of the present invention.

There is a need for a topical application of steroids that avoids the problems described above.

### SUMMARY OF THE INVENTION

An objective of the present invention is to provide a topical steroid composition that avoids application to healthy skin, such as a hand.

Another objective of the present invention is to provide a topical steroid composition that provides suitable penetration of the steroid into the skin to be treated.

The above objectives and other objectives are obtained by the present invention. The present invention provides a substantially water-free solid stick composition containing a corticosteroid where surprisingly the onset of action in clinical studies is faster than for conventional semisolid ointment compositions free of waxes.

The invention provides a solid stick topical composition comprising a corticosteroid present in a pharmaceutically effective amount, at least one petrolatum, at least one synthetic wax present in an amount sufficient such that the stick is solid at 25° C., propyleneglycol in an amount of about 5% to about 20% by weight, and an emulsifier present in an amount to emulsify the propyleneglycol and petrolatum. The solid stick is contained in a protective covering so that during application it does not come in contact with the hand holding the stick.

The invention also provides a method of making a solid stick comprising the steps of combining at least one petrolatum, at least one wax, propyleneglycol in an amount of about 5% to about 20% by weight; and an emulsifier present in an amount to emulsify the propyleneglycol and petrolatum to form a topical composition, heating the topical composition to a temperature sufficient to melt the wax, mixing the heated topical composition, pouring the heated topical composition into a mould in the shape of a solid stick, allowing the topical composition to cool to form a solid stick, and packaging the solid stick in an outer protective covering, or pouring the heated topical composition into the outer protective covering and allowing the heated topical composition to cool to form the solid stick in the outer protective covering, wherein the solid stick is substantially water free, and adding at least one corticosteroid in a pharmaceutically effective amount to the topical composition before, during or after the step of heating the topical composition.

The invention further provides a method of applying a corticosteroid to skin to treat a dermatological disease comprising the steps of opening an outer protective covering to reveal a solid stick, and rubbing the solid stick on a skin surface to be treated for a dermatological disease whereby the solid stick does not come in contact with skin on a hand holding the outer protective covering, and wherein the solid stick is formed from a topical composition comprising at least one corticosteroid present in a pharmaceutically effective amount, at least one petrolatum, at least one wax present in an amount sufficient such that the composition is solid at 25° C., propyleneglycol in an amount of about 5% to about 20% by weight, and an emulsifier present in an amount to emulsify the propyleneglycol and petrolatum.

### BRIEF DESCRIPTION OF THE DRAWINGS

FIG. 1 illustrates a graph of the results of the dynamic vasoconstriction study conducted in the Examples; and

FIG. 2 illustrates a view of a stick according to the present invention.

### DETAILED DESCRIPTION OF THE INVENTION

The present composition comprises a propyleneglycol, a petrolatum, a wax, an emulsifier and an active component in the form of a corticosteroid.

The amount of propylene glycol in the formulation will affect not only the solubility but also the rate of penetration of the drug through the skin. In general, the greater amount of propylene glycol the higher the solubility and faster the rate of the penetration. Suitable penetration rates have been found by using propylene glycol in an amount of about 5 to about 20% by weight, preferably about 10 to about 20% by weight. All amounts disclosed herein are weight percent based on the total weight of the composition unless otherwise stated. It has been found that for the particular corticosteroid, Clobetasol propionate, the amount of propylene glycol is preferably from about 10 to about 13%, and the most preferred range is 12 to 13%. The amount of propylene glycol may vary slightly for different corticosteroids.

The most dominant excipient in the formulation as been found to be petrolatum, also commonly referred to as petroleum jelly. Petrolatum is usually obtained as the semisolid residue from petroleum after the lighter and more volatile components have been boiled off. The Handbook of Pharmaceutical Exipients, American Pharmaceutical Association, Washington (Editors Ainley Wade and Paul J Weller) Second Edition 1994, defines petrolatum as a purified mixture of semisolid saturated hydrocarbons having the general formula C(n)H(2n+2) obtained from petroleum. The hydrocarbons comprise mainly branched and unbranched chains, although some cyclic alkanes and aromatic molecules with paraffin side chains may also be present. The term petrolatum also includes other mixtures of petroleum based semisolid hydrocarbons that are commonly referred to in the art or commercially sold as “petrolatum” or “petroleum jelly” that are suitable for application to skin. A common petrolatum is sold under the trademark Vaseline.

Without being bound to any particular theory of operation, petrolatum is believed to be effective in the composition as an unguentous mass and acts in the skin as an emollient. It is also believed that the petrolatum further functions as a skin repair initiator. Petrolatum can be included in amounts ranging up to about 80% by weight, preferably from about 50 to about 80%, and more preferably from about 60 to about 80% by weight. Some of the petrolatum may be replaced with other lipids or combinations of lipids. Thus, the term unguentous mass includes pure petrolatum or mixtures of lipids with similar properties with respect to solubility, lipophilicity and melting point. In the examples disclosed herein, a white petrolatum from Witco, Germany has been utilized. If petrolatum from other manufacturers is used in the inventive formulation, the amounts disclosed herein may have to be slightly altered in order to achieve the desired properties of the formulation, which is well within the skill of one of ordinary skill in the art. Examples of other commercially available petrolatum include, but are not limited to, those from Ultra Chemical, Carolina Medical Products and Amco Chemical.

The amount of wax(es) used depends on the choice of wax or mixture of waxes. The wax should be selected and present in an amount to provide a solid stick that substantially retains its structure at 25° C., and preferably at 35° C. Suitable waxes comprise synthetic waxes, as well as glycerol or glycol esters of fatty acids having an average carbon chain length of 18 to 36. The wax is usually provided in an amount of about 10 to about 20% by weight to provide a solid stick having desired properties. Waxes with the brand name Syncrowax have been used in the examples disclosed herein. It is of course possible to utilize waxes with similar properties from other manufacturers. The paraffin wax may differ in properties but to our knowledge any quality can be used although some qualities may induce minor changes to the composition that one of ordinary skill in the art would be able to accomplish without undue experimentation.

An emulsifier is included in the formulation since the solubility of propyleneglycol in petrolatum is usually lower than the maximum added amount. The emulsifier should be added in an amount that suitably emulsifies the propyleneglycol and petrolatum. Preferably, the emulsifier is present in an amount that keeps the composition more translucent at lower temperatures than if not added, which is important in the packaging procedure. Thus, the amount of emulsifier used will depend on the amount of propyleneglycol included. Preferably, the ratio between the propyleneglycol and emulsifier is from about 8:1 to about 12:1 v/v for liquids and v/w for solid emulsifiers. A preferred emulsifier is sorbitan sesquioleate but other emulsifiers that are suitable for emulsifying the propylene glycol and petrolatum may be used if desired.

The term “substantially water-free” means very small amounts of water that are usually introduced into the composition along with the components and manufacturing process, such as less than 2%, preferably less than 1% by weight based on the weight of the total composition. Propyleneglycol is allowed to contain 0.2% water according to U.S. Pat. No. 25, and thus a small amount of water will usually be introduced into the composition along with the propyleneglycol. Water may also be introduced into the composition by precipitation during the cooling phase of manufacturing. The presence of water, especially at 2% and greater, has negative effects on the stability of the product since it will effect the solubility of propyleneglycol in the base, and thus the amount of water should be maintained below 2% and preferably less than 1%. Most preferably, no water is intentionally added to the composition. Other agent's intended for enhancing cosmetic properties, such as oleyl alcohol, as well as conventional additives, may be added in limited amounts, for example, up to about 10% by weight.

Any corticosteroid used for topical application can be used in the present composition. Preferred examples of corticosteroids are the commonly referred to very potent corticosteroids and potent to medium potent corticosteroids. Examples of suitable very potent corticosteroids include, but are not limited to, Clobetasol propionate, Halcinonide and Diflucortolone valerate. Examples of suitable potent to medium potent corticosteroids include, but are not limited to, Triamcinolone acetonide, Betamethasone valerate, Fluticasone valerate, betamethasone dipropionate, Mometasone furoate, Hydrocortisone-17-butyrate, Beclomethasone dipropionate and Floucinolone acetonide. A preferred corticosteroid is Clobetasol propionate.

The corticosteroid can be present in a pharmaceutically effective amount. Examples of suitable amounts for the preferred corticosteroid, Clobetasol propionate, are from about 0.01 to about 1% by weight. Other types of corticosteroids may be used in different amounts and, based upon the disclosure provided herein, one skilled in the art of formulating will easily be able to determine a suitable amount to be used in a stick composition according to the present invention.

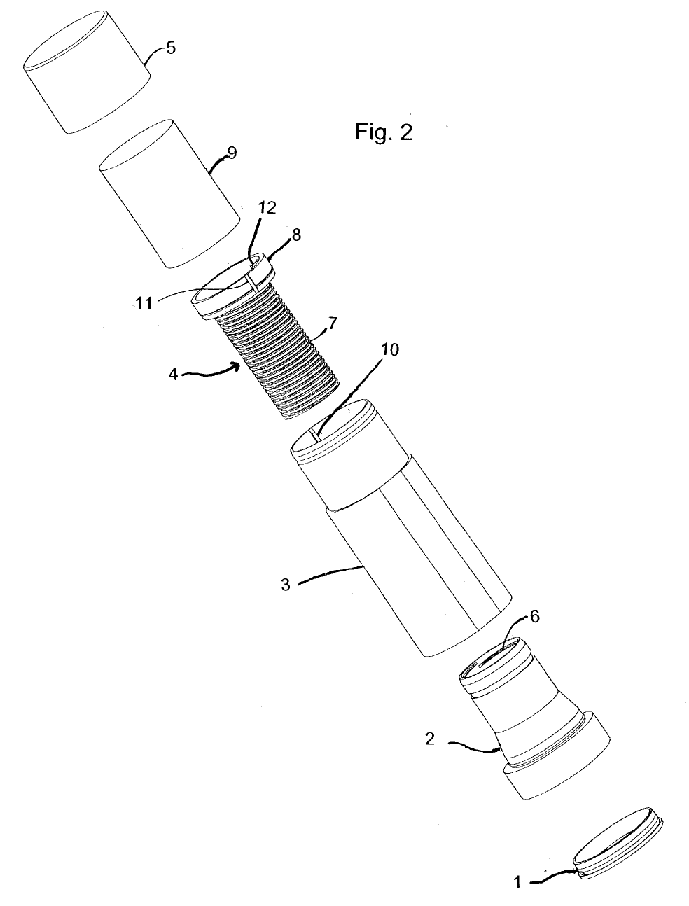

As shown in FIG. 2, the stick comprises the formulation 1 contained by a protective outer structure 2 that prevents contact between a hand and the active composition during application. While any suitable material can be used, preferred materials are polyethylene and polypropylene.

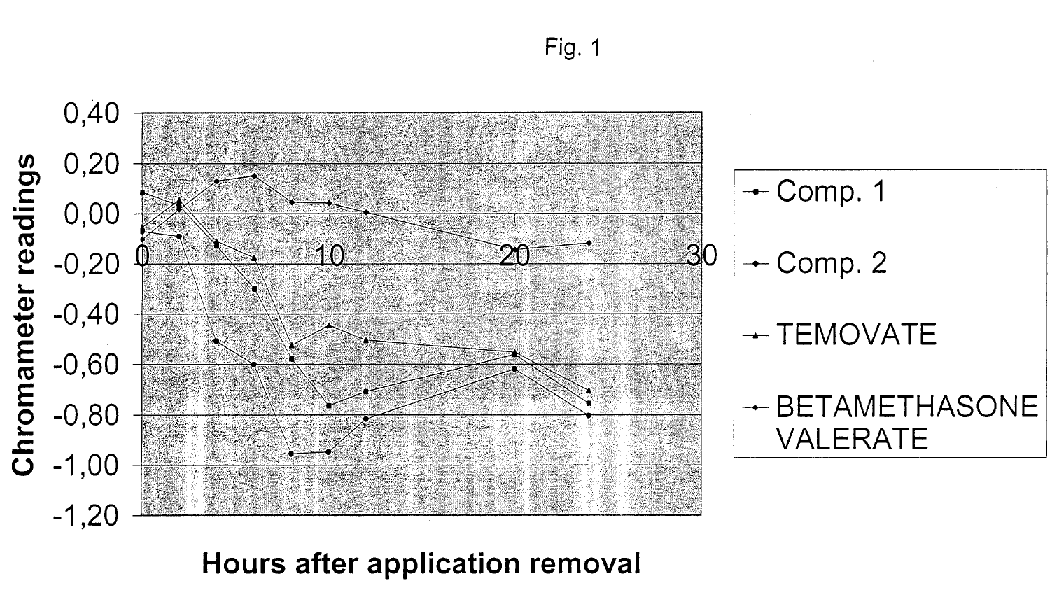

An example of a suitable protective covering is shown in FIG. 1. This protective covering is formed from polyethylene MD, and has no membrane. The protective covering comprises five parts: bottom lid 1, screw attachment 2, body 3, screw 4, and cap 5. All materials except for the bottom lid 1 and screw attachment 2 are coloured white by using titanium dioxide and calcium carbonate. The bottom lid 1 and screw attachment 2 is coloured blue by the addition of I.C.Pigment Blue 15:3 and C.I. Pigment Green 7. The bottom lid 1 is removed during packing when the melted material is dispensed though the hollow screw attachment 2 and the screw 4. The cylindrical shaped screw attachment 2 has threads 6 on the inner surface while the screw 4 is covered with threads 7 on the outer periphery. The top of the screw 4 has a horizontal section 8 where the stick 9 is attached. By rotating the screw attachment 2 the stick 9 is transported upwards. The body 3 is equipped with guides 10 and corresponding splines 11 on the screw 4 prevents the stick 9 from rotating. The screw 4 contains two splines 12 to give the stick 9 an attachment surface. The stick is currently envisioned in two sizes: 1) a 50 ml size of about 120 mm in height by 42 mm in diameter, and 2) a 20 ml size of about 111 mm in height and about 32 mm in diameter. However, the stick can be formed to any size for the desired application.

Manufacture

The stick formulations can be manufactured by combining the components to form a topical composition, heating the topical composition to a temperature which melts the wax, such as about 70° C. to about 80° C., stirring to dissolution, and then packaging the topical composition in the outer protective covering. The packaging can be conducted at a lower temperature, such as about 50° C. to about 60° C., with 50 to 55° C. being preferred. The warm packages can be allowed to cool slowly to ambient temperature, which allows the topical composition to harden thereby forming the solid stick. Alternatively, the heated topical composition can be poured into moulds and allowed to cool to form the solid sticks. The solid sticks can then be packaged in the outer protective covering. If desired, the corticosteroid can be combined with the remaining components before, during or after the step of heating. If the corticosteroid is heat sensitive at the temperature the topical composition is heated to, the corticosteroid can be added to the topical composition during the cooling step.

### EXAMPLES

### Example 1

Two formulations of the inventive formulation were manufactured according to the mentioned manufacturing method.

TABLE 1
Stick compositions

Amount (% w/w)

| Ingredient | Composition 1 | Composition 2 |
|---|---|---|
| Syncrowax ERLC | 3 | 3 |
| Syncrowax HGLC | 12 | 12 |
| Petrolatum (Witco) | 79.45 | 66.2 |
| Propyleneglycol | 5 | 12.5 |
| Sorbitan sesquioleate | 0.5 | 1.25 |
| Oleyl alcohol | — | 5 |
| Clobetasol propionate | 0.05 | 0.05 |
| Sum | 100 | 100 |

The formulations were manufactured by careful mixing and heating to 75° C. After completion of mixing the temperature was cooled to 60° C. and the melted stick mass was packed in preheated stick protective covers. The protective covers were allowed to cool slowly after filling.

The sticks were tested for stability with respect to several physical and chemical parameters. A penetrometer test was conduced to determine the hardness of the stick according to ASTM1321/DIN 51579. The stick was a solid at room temperature (25° C.) as well as at 35° C. This means that the stick will not spread or run on the skin and consequently will remain within the area of application.

Comparison of Example 1 Stick Compositions 1 and 2 to Conventional Topical Corticosteroid

The vasoconstrictive effect of a topical corticosteroids is often used for the evaluation of the clinical effect during development work since vasoconstriction is regarded to be a good indicator of clinical effect. In this experiment we have used a dynamic vasoconstriction method according to Stauton and McKenzie and the number of healthy volunteers included was 20. The preparations were applied for 45 minutes, after which time the preparations were removed and vasoconstriction, blanching, was determined by a cromameter for 6 hours. The compositions studied were Composition 1, Composition 2 and a commercially available ointment Temovate. A lower potency comparator, betametasone valerate USP ointment, was included as well. The principal composition of Temovate is listed in table 2.

TABLE 2
Ointment composition

| | Amount (% w/w) |
|---|---|
| Ingredient | Temovate ® ointment |
| Petrolatum | 94.45 |
| Propyleneglycol | 5.0 |
| Sorbitan sesquioleate | 0.5 |
| Clobetasol propionate | 0.05 |

In FIG. 1 and in table 3 the results of the dynamic vasoconstriction study is presented.

TABLE 3
Results of vasoconstriction,
mean cromameter readings after 45 minutes of application.

| | Composition | Composition | | Betametasone |
|---|---|---|---|---|
| Hours | 1 | 2 | TEMOVATE | valerate USP |
| 0 | 0.09 | −0.07 | −0.06 | −0.10 |
| 2 | 0.03 | −0.09 | 0.06 | 0.02 |
| 4 | −0.13 | −0.51 | −0.11 | 0.13 |
| 6 | −0.30 | −0.60 | −0.18 | 0.15 |
| 8 | −0.58 | −0.96 | −0.52 | 0.05 |
| 10 | −0.76 | −0.95 | −0.45 | 0.04 |
| 12 | −0.71 | −0.82 | −0.51 | 0.01 |
| 20 | −0.56 | −0.62 | −0.55 | −0.14 |
| 24 | −0.76 | −0.80 | −0.71 | −0.12 |

AUC was determined as well and the results are demonstrated in table 4.

TABLE 4
AUC after 45 minutes of application.

| Composition | 1 | 2 | Temovate | BMV |
|---|---|---|---|---|
| AUC_{(45 min)} | −11.77 | −15.65 | −9.67 | −0.41 |

The data indicates that the amount of corticosteroid that has penetrated into the skin was remarkably higher for the two stick formulations according to the present invention than for the corresponding conventional ointment formulations. This is an unexpected effect since it is well known that an increase of viscosity in a topical vehicle normally has a negative effect on the skin penetration rate of the included drug.

While the claimed invention has been described in detail and with reference to specific embodiments thereof, it will be apparent to one of ordinary skill in the art that various changes and modifications can be made to the claimed invention without departing from the spirit and scope thereof.

1. A solid stick comprising:
a topical composition in the form of a solid stick comprising,
at least one corticosteroid present in a pharmaceutically effective amount;
at least one petrolatum;
at least one wax present in an amount sufficient such that the composition is solid at 25° C.;
propyleneglycol in an amount of about 5% to about 20% by weight; and an emulsifier present in an amount to emulsify the propylene glycol and petrolatum; and
an outer protective covering containing the solid stick and being constructed and arranged to prevent contact between a hand holding the outer protective covering and the solid stick, wherein the topical composition is substantially water-free.

2. A solid stick according to claim 1, wherein the wax is present in an amount of about 10 to about 20% by weight based on the total weight of the composition.

3. A solid stick according to claim 1, wherein the wax comprises at least one selected from the group consisting of C18 to C36 acid glycerol or glycol esters.

4. A solid stick according to claim 1, wherein the wax is present in an amount sufficient such that the stick is solid a 35° C.

5. A solid stick according to claim 1, wherein the wax is present in such an amount that the stick is a solid and substantially retains its shape at 25° C.

6. A solid stick according to claim 1, wherein the wax comprises a soft white paraffin.

7. A solid stick according to claim 1, wherein propyleneglycol is present in an amount of about 10 to about 20% by weight based on the total weight of the composition.

8. A solid stick according to claim 1, wherein propyleneglycol is present in an amount of about 10 to about 13% by weight based on the total weight of the composition.

9. A solid stick according to claim 1, wherein propyleneglycol is present in an amount of about 12 to about 13% by weight based on the total weight of the composition.

10. A solid stick according to claim 1, wherein the petrolatum is present in an amount of about 50 to about 80% by weight based on the total weight of the composition.

11. A solid stick according to claim 1, wherein the petrolatum is present in an amount of about 60 to about 80% by weight based on the total weight of the composition.

12. A solid stick according to claim 1, further comprising a lipid.

13. A solid stick according to claim 1, wherein the emulsifier comprises sorbitan sesquioleate.

14. A solid stick according to claim 1, wherein emulsifier is present in an amount of about 0.2 to about 2.0% by weight based on the total weight of the composition.

15. A solid stick according to claim 1, wherein the ratio between propyleneglycol:emulsifier is from about 8:1 to about 12:1 v/v for liquids and v/w for solid emulsifiers.

16. A solid stick according to claim 1, wherein the corticosteroid is present in an amount of about 0.01 to about 1% by weight based on the total weight of the composition.

17. A solid stick according to claim 1, wherein the corticosteroid comprises Clobetasol propionate.

18. A solid stick according to claim 1, wherein the corticosteroid comprises at least one selected from the group consisting of Clobetasol propionate, Halcinonide, Diflucortolone valerate, Triamcinolone acetonide, Betamethasone valerate, Fluticasone valerate, betamethasone dipropionate, Mometasone furoate, Hydrocortisone-17-butyrate, Beclomethasone dipropionate and Floucinolone acetonide.

19. A solid stick according to claim 1, wherein the topical composition contains less than 2% by weight water.

20. A solid stick according to claim 1, wherein the topical composition contains less than 1% by weight water.

21. A stick according to claim 1, wherein the outer protective covering comprises a body and a mechanism to lower and raise the solid stick from the body.

22. A stick according to claim 21, wherein the outer protective covering further comprises a screw attachment and screw being constructed and arranged such when the screw is turned in relation to the screw attachment the screw raises or lowers in relation to the body, and the screw comprises a horizontal section for holding the stick.

23. A topical composition for forming a solid stick comprising,
at least one corticosteroid present in a pharmaceutically effective amount;
at least one petrolatum;
at least one wax present in an amount sufficient such that the composition is solid at 25° C.;
propyleneglycol in an amount of about 5% to about 20% by weight; and an emulsifier present in an amount to emulsify the propylene glycol and petrolatum, wherein the topical composition is substantially water-free.

24. A method of making a solid stick comprising the steps of:
combining at least one petrolatum, at least one wax; propyleneglycol in an amount of about 5% to about 20% by weight; and an emulsifier present in an amount to emulsify the propylene glycol and petrolatum to form a topical composition;
heating the topical composition to a temperature sufficient to melt the wax;
mixing the heated topical composition;
pouring the heated topical composition into a mould in the shape of a solid stick, allowing the topical composition to cool to form a solid stick, and packaging the solid stick in an outer protective covering, or pouring the heated topical composition into the outer protective covering and allowing the heated topical composition to cool to form the solid stick in the outer protective covering, wherein the solid stick is substantially water free; and
adding at least one corticosteroid in a pharmaceutically effective amount to topical composition before, during or after the step of heating the topical composition.

25. A method of applying a corticosteriod to skin to treat a dermatological disease comprising the steps of:
opening an outer protective covering to reveal a solid stick; and
rubbing the solid stick on a skin surface to be treated for a dermatological disease whereby the solid stick does not come in contact with skin on a hand holding the outer protective covering, and wherein the solid stick is formed from a topical composition comprising at least one corticosteroid present in a pharmaceutically effective amount, at least one petrolatum, at least one wax present in an amount sufficient such that the composition is solid at 25° C., propyleneglycol in an amount of about 5% to about 20% by weight, and an emulsifier present in an amount to emulsify the propylene glycol and petrolatum.
